(19) United States
(12) Patent Application Publication
(10) Pub. No.: US 2008/0249529 A1
(43) Pub. Date: Oct. 9, 2008

(54) Dual incision disc repair device and method

(75) Inventors: Brett R. Zarda, Providence, RI (US); Christine Rusbarsky, Norton, MA (US); Andrew P. Dooris, Raynham, MA (US); Mark T. Hall, Bridgewater, MA (US)

Correspondence Address: MAGINOT, MOORE & BECK, LLP;CHASE TOWER, 111 MONUMENT CIRCLE, SUITE 3250, INDIANAPOLIS, IN, 46204, US

(73) Assignee: DePuy Spine, Inc., Raynham, MA (US)

(21) Appl. No.: 11/724,381

(22) Filed: Mar. 15, 2007

(51) Int. Cl. A61B 17/58; A61B 19/00; A61B 17/32; A61M 29/00

(52) U.S. Cl. 606/93; 128/898; 606/167; 606/192

(57) ABSTRACT

The invention includes a device and method for positioning the device within a disc space of a patient. In one embodiment, a method of positioning an implant within a disc space of a patient includes making a first incision in an annulus of an intervertebral disc, making a second incision in the annulus of the intervertebral disc, clearing a space within the intervertebral disc, moving a first filler portion into the space through the first incision, moving a second filler portion into the space through the first incision and moving the second filler portion out of the space through the second incision.

### FIELD OF THE INVENTION

This invention relates to surgical procedures and devices and, more particularly, to methods and devices for repairing intervertebral discs in a surgical patient.

### BACKGROUND

The spinal column acts as a major structural support. Various mechanisms, however, affect the ability of intervertebral discs to provide the requisite stability and support. For example, the normal aging process tends to weaken the bones and tissues associated with the spinal column increasing the risk of spinal injuries. Additionally, sudden movements may cause a disc to rupture or herniate. A herniation of the disc is primarily a problem when the nucleus pulposus protrudes or ruptures into the spinal canal placing pressure on nerves which in turn causes spasms, tingling, numbness, and/or pain in one or more parts of the body, depending on the nerves involved. Further deterioration of the disc can cause the damaged disc to lose height and to produce bone spurs. These mechanisms may result in a narrowing of the spinal canal and foramen, thereby causing undesired pressure on the nerves emanating from the spinal cord.

Treatments of spinal cord conditions include various procedures which involve the removal of all or a portion of a spinal component. Such procedures may include the injection of an enzyme into an affected disc to dissolve tissues. The enzymes typically used in this procedure are protein-digesting enzymes which must be carefully placed with respect to the spinal defect to avoid inadvertent dissolution of spinal tissue.

Alternatively, surgical access to a spinal area may be obtained and a tool such as a curette, osteotome, reamer, rasp, or drill may be used to mechanically reshape a component of the spinal column. The tissue removed may include disc tissue which is causing pressure on a nerve or the spinal canal. This technique is highly invasive and traumatic to the body, and therefore requires an extended recovery period. Moreover, there are increased risks of future problems due to the removal of a portion of the lamina which is no longer in place to support and protect the spinal canal at the area where the surgery took place.

Surgical access may also be used for spinal fusion surgery. In a fusion procedure, a damaged disc may be completely removed. Parts of a bone from another part of the patient's body, such as the pelvis, are harvested, and the bone parts or grafts are subsequently placed between the adjacent vertebrae so that the adjacent vertebrae grow together in a solid mass. The recovery time for a normal spinal fusion surgery is significant due not only to the fact that normal movement cannot be allowed until detectable bone growth has occurred between the bone grafts and the adjacent vertebrae, but also due to the fact that the associated ligaments and muscles, both at the spinal location and the location where the bone grafts were harvested, must also recover.

Recently, efforts have been directed to replacing defective spinal column components, specifically, all or portions of the intervertebral disc. When this type of procedure is performed in a minimally invasive manner, it is known for various devices implanted during the procedure to be subsequently expelled from the intervertebral discs. This expulsion is frequently attributed to inadequate clearance of the nucleus during the minimally invasive surgical procedure. Alternatively, normal biomechanical motion places large stresses upon the nucleus which can force migration and ultimately expulsion of the device through the compromised annulus. The result is that the implanted device extrudes from the cavity formed in the spinal column, increasing the potential for clinical complications.

A need exists for a method and device that is minimally invasive, easy to use, and safe. A further need exists for a method and device that reduces the risk of expulsion of the device. A further need exists for a method and device which provides timely indication of the position of the device.

### SUMMARY

A filler and method for implanting a filler within a disc space is disclosed. In one embodiment according to the invention, a method of positioning an implant within a disc space of a patient includes making a first incision in an annulus of an intervertebral disc, making a second incision in the annulus of the intervertebral disc, clearing a space within the intervertebral disc, moving a first filler portion into the space through the first incision, moving a second filler portion into the space through the first incision and moving the second filler portion out of the space through the second incision.

In accordance with another embodiment, a method of positioning an implant within a disc space includes making a first incision in an annulus of an intervertebral disc, making a second incision in the annulus of the intervertebral disc, clearing a space within the intervertebral disc, moving a first filler portion into the space through the first incision and determining the position of the first filler portion using the second incision.

In a further embodiment, a device implanted within an intervertebral disc includes a main body portion located within the intervertebral disc, a first lead connected to the main body portion and extending out of the intervertebral disc through a first incision and a second lead connected to the main body portion and extending out of the intervertebral disc through a second incision.

The above-described features and advantages, as well as others, will become more readily apparent to those of ordinary skill in the art by reference to the following detailed description and accompanying drawings.

### BRIEF DESCRIPTION OF THE DRAWINGS

FIG. 1 depicts a side perspective view of the spinal column of a human;

FIG. 2 depicts a coronal view of a lumbar vertebra, partially cut away and in section, taken generally along line A-A in FIG. 1;

FIG. 3 depicts a partial vertical cross-sectional view of lumbar vertebrae from the spinal column of FIG. 1;

FIG. 4 depicts the partial vertical cross-sectional view of the lumbar vertebrae of FIG. 3 showing the nucleus and annulus of the intervertebral disc;

FIG. 5 depicts a horizontal cross-sectional view of an intervertebral disc showing the nucleus and annulus of the intervertebral disc;

FIG. 6 depicts a cannula used to incise the annulus of the intervertebral disc of FIG. 5 using a posterior approach in accordance with principles of the invention;

FIG. 7 depicts a partial cross-sectional view of the sheath of the cannula of FIG. 6 showing an aspiration bore and a return bore adjacent to the inner bore of the cannula;

FIG. 8 depicts a cross-sectional view of the intervertebral disc of FIG. 5 and a partial perspective view of the cannula of FIG. 6 inserted within a first cavity within the intervertebral disc in accordance with principles of the invention;

FIG. 9 depicts a cross-sectional view of the intervertebral disc of FIG. 5 with a partial perspective view of the cannula of FIG. 6 and a partial perspective view of a second cannula used to incise the annulus and to form a second portion of the cavity in accordance with principles of the invention;

FIG. 10 depicts a partial cross-sectional view of two cannula extending through two incisions in the annulus of an intervertebral disc with a cavity formed therein using an anterior approach in accordance with principles of the invention;

FIG. 11 depicts the intervertebral disc of FIG. 10 with a filler device made from a in-situ curable material partially positioned within the cavity through a first cannula in accordance with principles of the invention;

FIG. 12 depicts the filler device of FIG. 11 completely filling the cavity of the intervertebral disc and extending into the second cannula;

FIG. 13 depicts a perspective view of an alternative filler device with an inflatable main body portion and two leads in accordance with principles of the invention;

FIG. 14 depicts a perspective view of the filler device of FIG. 13 in a deflated or deformed condition;

FIG. 15 depicts a perspective view of the filler device of FIG. 13 with a leading lead positioned through a first cannula, through a cavity in an intervertebral disc and through a second cannula in accordance with principles of the invention;

FIG. 16 depicts the filler device of FIG. 13 with the main body portion positioned within the cavity of the intervertebral disc, a trailing lead extending out of the first cannula and the leading lead extending out of the second cannula;

FIG. 17 depicts the filler device of FIG. 13 expanded within the cavity of the intervertebral disc;

FIG. 18 depicts a partial perspective view of two leads extending posteriorly out of an intervertebral disc and affixed to a vertebra in accordance with principles of the invention;

FIG. 19 depicts a partial perspective view of two leads extending posteriorly out of an intervertebral disc and affixed to a each other in accordance with principles of the invention;

FIG. 20 depicts a partial perspective view of four leads extending anteriorly out of an intervertebral disc and affixed to two adjacent vertebrae in accordance with principles of the invention;

FIG. 21 depicts a partial perspective view of a single lead extending anteriorly out of an intervertebral disc and affixed to a vertebra in accordance with principles of the invention;

FIG. 22 depicts a partial cross-sectional view of two cannula extending through two incisions of different sizes in the annulus of an intervertebral disc with a cavity formed therein using an anterior approach in accordance with principles of the invention;

FIG. 23 depicts a perspective view of an alternative filler device with a deformable main body portion including a rigid disc portion in a deflated or deformed condition and two leads in accordance with principles of the invention;

FIG. 24 depicts the filler device of FIG. 23 with a leading lead positioned through the first cannula, the cavity in the intervertebral disc and the second cannula of FIG. 22 in accordance with principles of the invention; and

FIG. 25 depicts the filler device of FIG. 23 with the main body portion positioned within the cavity of the intervertebral disc and the rigid disc portion abutting the second cannula, a trailing lead extending out of the first cannula and the leading lead extending out of the second cannula.

### DETAILED DESCRIPTION

FIG. 1 depicts a spinal column 100 which includes a number of vertebrae 102, a sacrum 104, and a coccyx 106. The number of vertebrae 102 that make up the spinal column 100 depends upon the species. In a human (which FIG. 1 shows), there are typically twenty-four vertebrae 102 including seven cervical vertebrae 108, twelve thoracic vertebrae 110, and five lumbar vertebrae 112. When viewed from the side the spinal column 100 forms a sinusoidal pattern. The sinusoidal pattern serves to support the head.

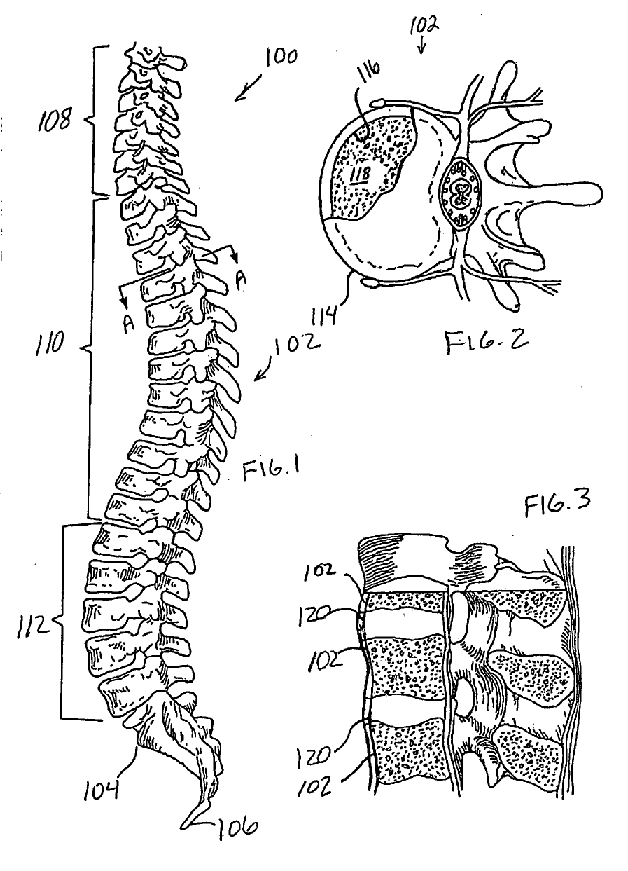

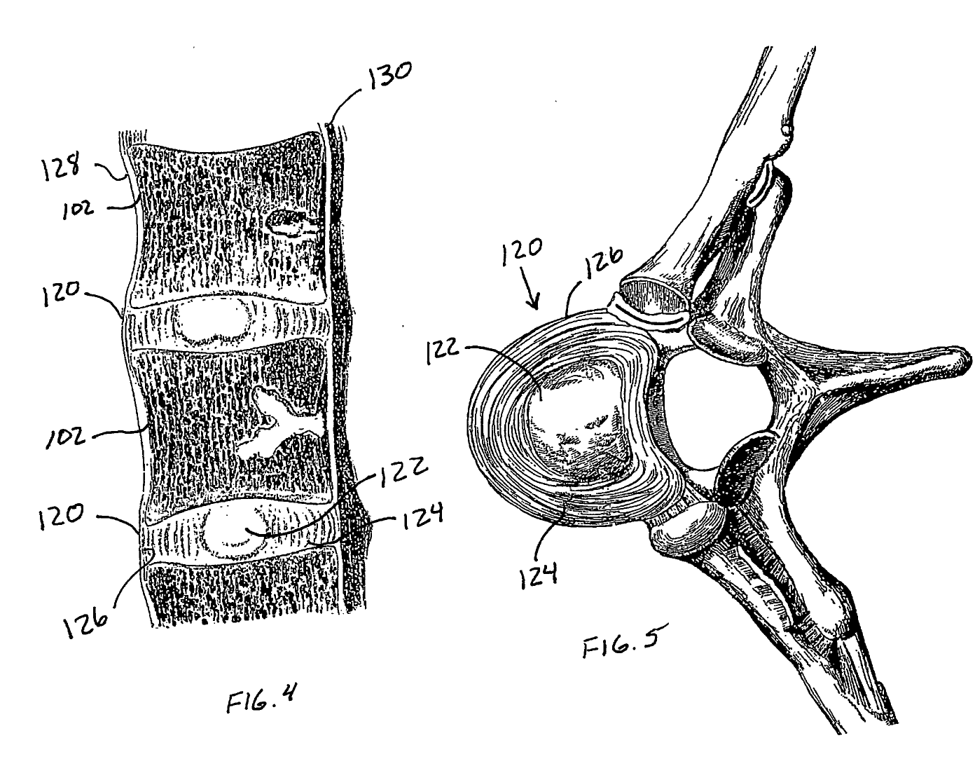

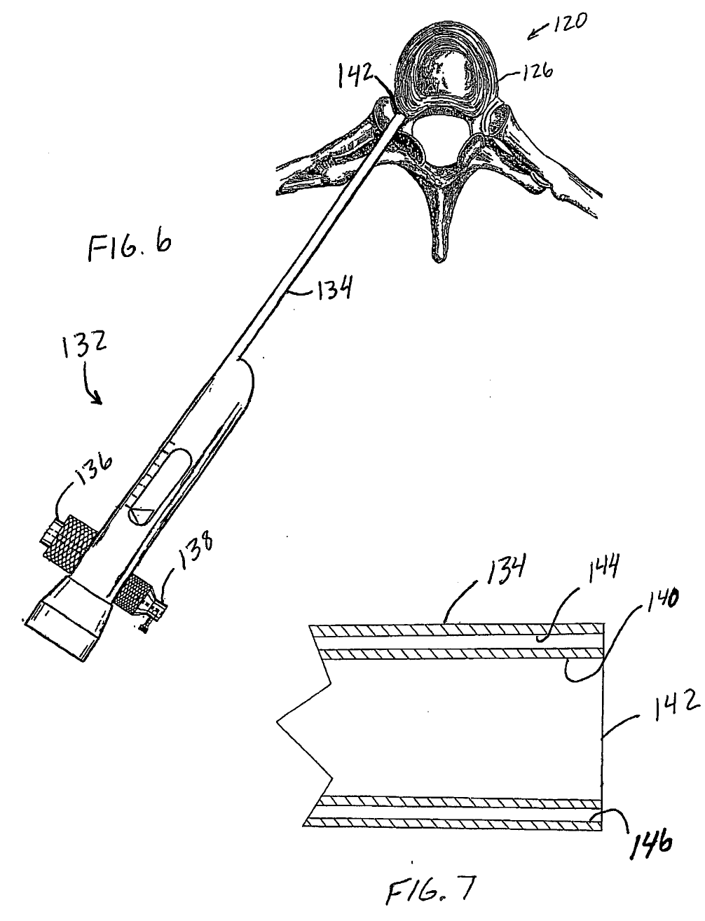

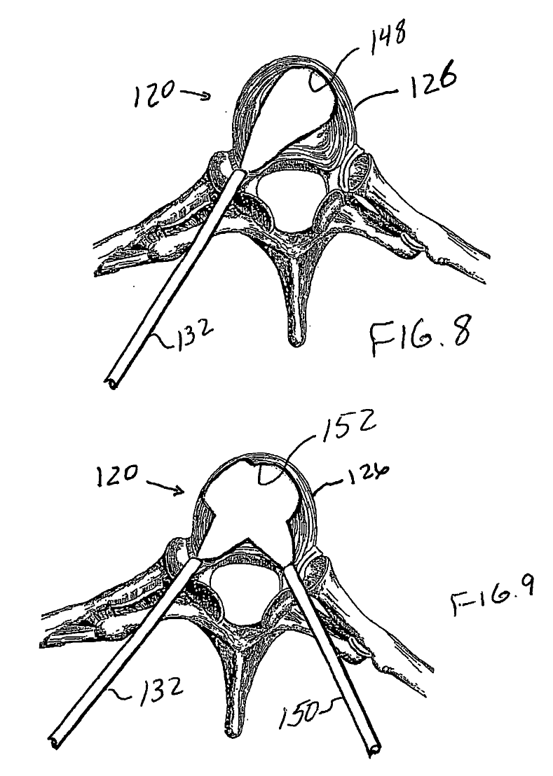

Each vertebra 102 includes a vertebral body 114, which extends on the anterior (i.e., front or chest) side of the vertebra 102 as shown in FIG. 2. The vertebral body 114 is in the shape of an oval disc. The vertebral body 114 includes an exterior formed from compact cortical bone 116. The cortical bone 116 encloses the medullary bone 118 which is a volume of reticulated, cancellous or spongy bone. As shown in FIG. 3, each vertebra 102 is separated from adjacent vertebrae 102 by an intervertebral disc 120.

FIGS. 4 and 5 show additional detail of the intervertebral discs 120 of FIG. 3. The intervertebral discs 120 provide the chief bond of connection between the adjacent vertebrae 102. The intervertebral discs 120 vary in shape, size and thickness in different parts of the spinal column 100. The intervertebral discs 120 correspond in shape to the vertebrae 102 between which they are placed. In each of the intervertebral discs 120, a soft pulpy center 122 is surrounded by concentric laminae of fibro-cartilage 124. The outermost lamina, annulus 126, is a lamina of fibrous tissue. The annulus 126 is closely connected to the anterior common ligament 128 and posterior common ligament 130.

In an exemplary operation, a surgical site is prepared in an acceptable manner and the intervertebral disc 120 is exposed. One or more surgical sites may be selected so as to provide an anterior approach, a posterior approach, a bilateral approach or any other desired approach or combination of approaches. In this embodiment, a posterior approach has been selected. A cannula 132 is used to incise the annulus 126 of the intervertebral disc 120 as shown in FIG. 6. The cannula 132 includes a sheath 134, an inlet port 136 and an outlet port 138. The sheath 134 includes an internal bore 140 (see FIG. 7), a tip 142, a fluid supply bore 144 and a fluid return bore 146. The inlet port 136 is in fluid connection with the fluid supply bore 144 and may be attached to a fluid reservoir (not shown) to provide rinsing fluid to the cannula 132. The outlet port 138 is in fluid connection with the fluid return bore 146 and may be directed to a drain or collection system.

Once the tip 142 of the cannula 132 has incised the annulus 126, a cavity 148 may be formed in the intervertebral disc 120 as shown in FIG. 8. By way of example, an abrading member may be introduced into the intervertebral disc 120 through the internal bore 140 and used to loosen the center 122 and/or the laminae of fibro-cartilage 124. Rinse fluid may be introduced to the cavity 148 through the fluid supply bore 144 either contemporaneously with or subsequent to loosening of the tissue. The loosened tissue is then directed through the fluid return bore 146 and the outlet port 138 to the desired receptacle. If desired, a vacuum source may be applied to the outlet port 138 to assist in removal of loosened tissue and rinse fluid.

With reference to FIG. 9, a second cannula 150, which may be configured in the same manner as the cannula 132, is then used to make a second incision into the intervertebral disc 120 through the annulus 126. The second cannula 150 may be used to enlarge the cavity 148 as depicted by the cavity 152 in FIG. 9.

The foregoing steps may be modified in a number of ways. By way of example, the second cannula 150 may be inserted within the intervertebral disc 120 prior to formation of the cavity 148 within the intervertebral disc 120. Thus, the second cannula may be used to remove tissue loosened by the cannula 132. Moreover, a variety of abrading tools may be used so as to more closely conform the final cavity to the shape of the annulus 126. Additionally, while only two incisions are used in the foregoing example, additional incisions may be made into the intervertebral disc 120, including incisions from different approaches. These alternative embodiments may be selected based upon the particular needs of the patient.

Once the desired space has been obtained, a filler is introduced into the space. One method of introducing a filler into a cavity is explained with reference to the intervertebral disc 152 shown in FIG. 10. As depicted in FIG. 10, cavity 154 has been formed in the intervertebral disc 152 and a cannula 156 and a cannula 158 are positioned within two incisions 160 and 162, respectively, in the annulus 164 of the intervertebral disc 152. The cannula 156 includes an internal bore 166 and the cannula 158 includes an internal bore 168.

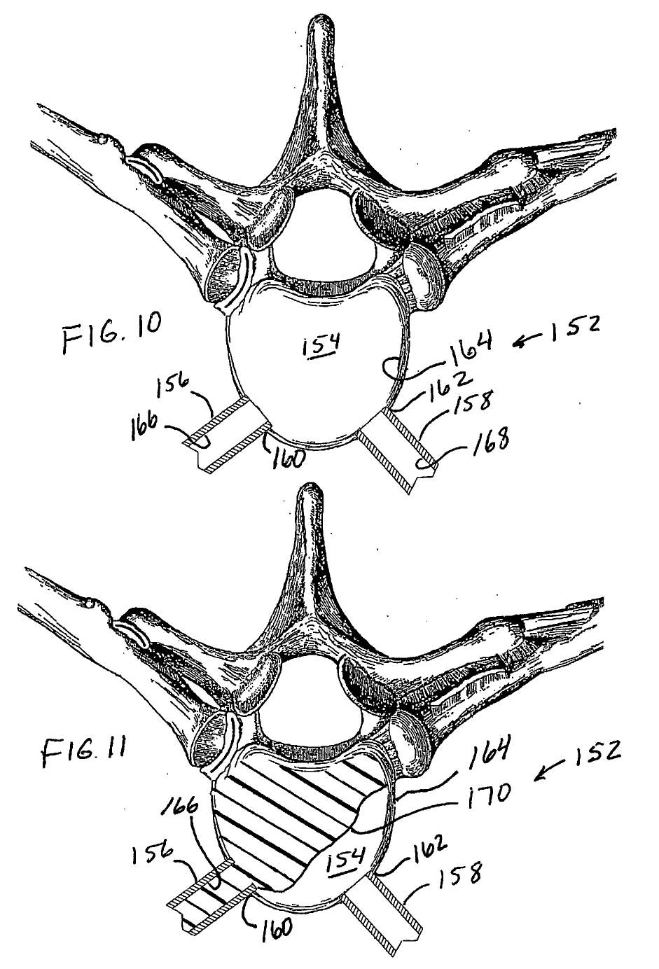

With reference to FIG. 11, an in-situ curable fluid 170 is introduced into the cavity 154 through the internal bore 166 of the cannula 156 which passes through the annulus 164 at the incision 160. The cannula 158 may be used to vent any fluids or materials within the cavity 154 during the fill procedure. When the cavity 154 is filled with the in-situ curable fluid 170, the in-situ curable fluid 170 begins to be extruded out of the cavity 154 through the incision 162 resulting in a protuberance 172 of in-situ curable fluid 170 within the internal bore 168 of the cannula 158 as shown in FIG. 12.

When the extrusion of the in-situ curable fluid 170 is detected, the introduction of in-situ curable fluid 170 into the cavity 154 through the cannula 156 may be terminated. Alternatively, the internal bore 168 may be plugged. This allows for the in-situ curable fluid 170 within the cavity 154 to be pressurized, thereby expanding the cavity. Once the desired amount of in-situ curable fluid 170 is located within the cavity 154, the in-situ curable fluid 170 is allowed to cure. Thereafter, the cannula 156 and 158 may be removed and any in-situ curable fluid 170 extending out of the cavity 154 through the incisions 160 and 162 may be removed.

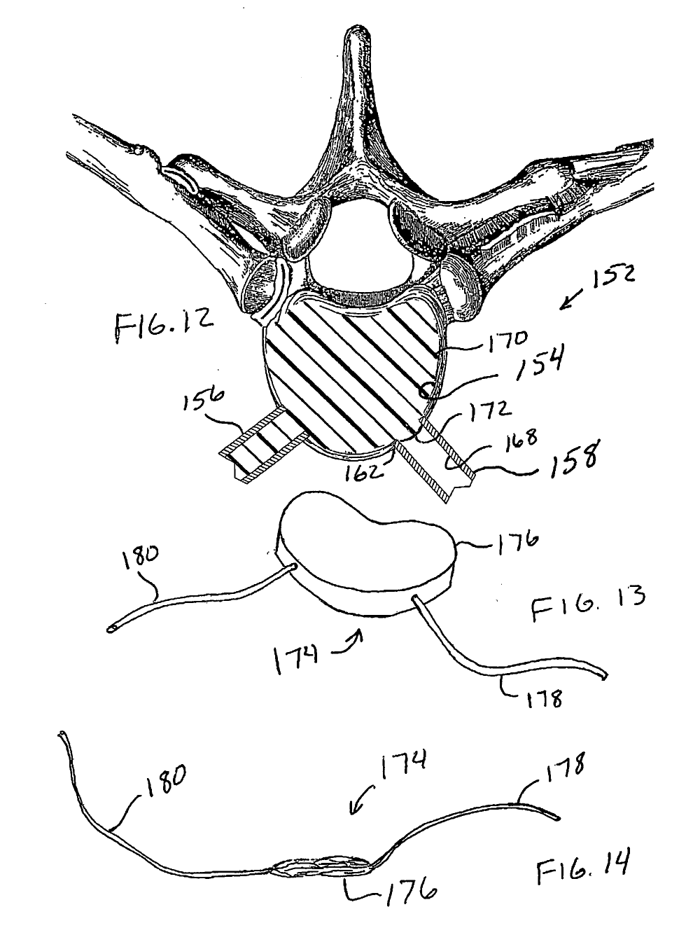

Deformable and/or inflatable fillers may also be introduced into an intervertebral disc cavity using two incisions in the intervertebral disc annulus. One such device is shown in FIG. 13. The filler device 174 includes a main body portion 176, and two leads 178 and 180. The main body portion 176 is hollow. Thus, the main body portion 176 may be deflated or deformed into a substantially cylindrical shape as shown in FIG. 14.

Insertion of the filler device 174 is explained with initial reference to FIG. 15, wherein a cavity 182 has been prepared in an intervertebral disc 184 and two cannula 186 and 188 are positioned through two incisions 190 and 192, respectively, through the annulus 194. Initially, the lead 178 is inserted through the cannula 186 past the incision 190 and the annulus 194 and into the cavity 182. The lead 178 is then threaded into the cannula 188 past the incision 192 and the annulus 194. In one embodiment, the lead 178 is threaded into the cannula 188 by inserting an instrument (not shown) into the cavity 182 through the cannula 188, grasping the lead 178 with the instrument (not shown) and then pulling the instrument (not shown) along with the grasped portion of the lead 178 outwardly from the cavity 182 into the cannula 188.

The main body portion 176 is then positioned within the cavity 182 as shown in FIG. 16 by manipulating the lead 178 and the lead 180. The positioning of the main body portion 176 within the cavity 182 may be ascertained in a variety of alternative manners. By way of example, one or both of the leads 178 and 180 may include markings (not shown) allowing the operator to determine the position of the main body portion 176. The markings may indicate the relative distance of the markings from the main body portion 176. Alternatively, the main body portion 176 may include a radiopaque indicator so that the operator can determine the positioning of the main body portion 176 using radiography.

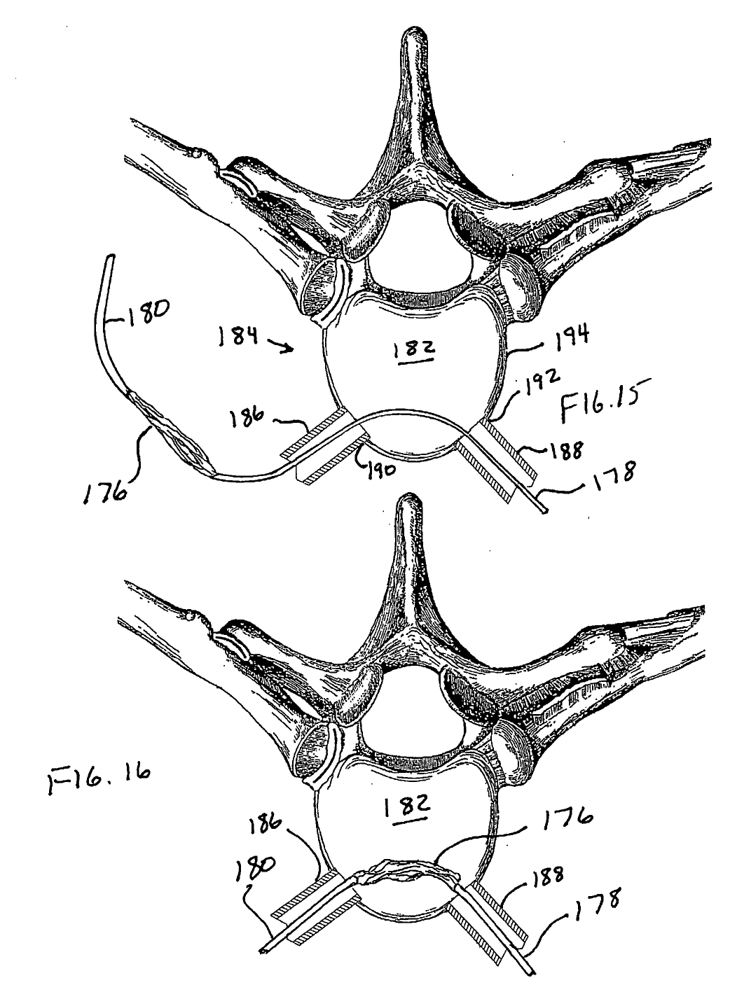

Once the main body portion 176 is in the desired position within the cavity 182, the main body portion 176 is inflated to the condition shown in FIG. 17. Inflation may be effected, for example, by inserting a needle (not shown) into the main body portion 176 through either of the cannula 186 and 188 and injection of a fluid into the main body portion 176 through the needle (not shown). Alternatively, one of both of the leads 178 and 180 may be provided with an internal bore (not shown) in fluid communication with the main body portion 176. Thus, the main body portion 176 may be inflated through the leads 178 and/or 180 with fluid from a fluid reservoir (not shown).

Once a filler device has been positioned, additional fixation may be accomplished using leads provided with the device. By way of example, FIG. 18 depicts an intervertebral disc 200 after a filler device has been positioned therein. Two leads 202 and 204 extend out of incisions 206 and 208, respectively, of an annulus 210 of the intervertebral disc 200. The leads 202 and 204 may either or both be provided as a single unit with the filler device. Alternatively, one or more leads may be coupled with the filler device during the procedure. By way of example, leads may be positioned partially within the cavity of an intervertebral disc which is filled with an in-situ curable material. Thus, as the material cures in-situ, the leads become coupled with the filler device. Alternatively, the leads may be attached in an acceptable manner after the material has cured.

Continuing with FIG. 18, the leads 202 and 204 are affixed to a vertebra 212 located adjacent to the intervertebral disc 200. Accordingly, the filler device is restrained from movement out of the intervertebral disc 200 through either the incision 206 or the incision 208. The leads 202 and 204 may be affixed to the vertebra 212 using a restraint device such as a screw, staple, nail, rivet, pin, glue or other device.

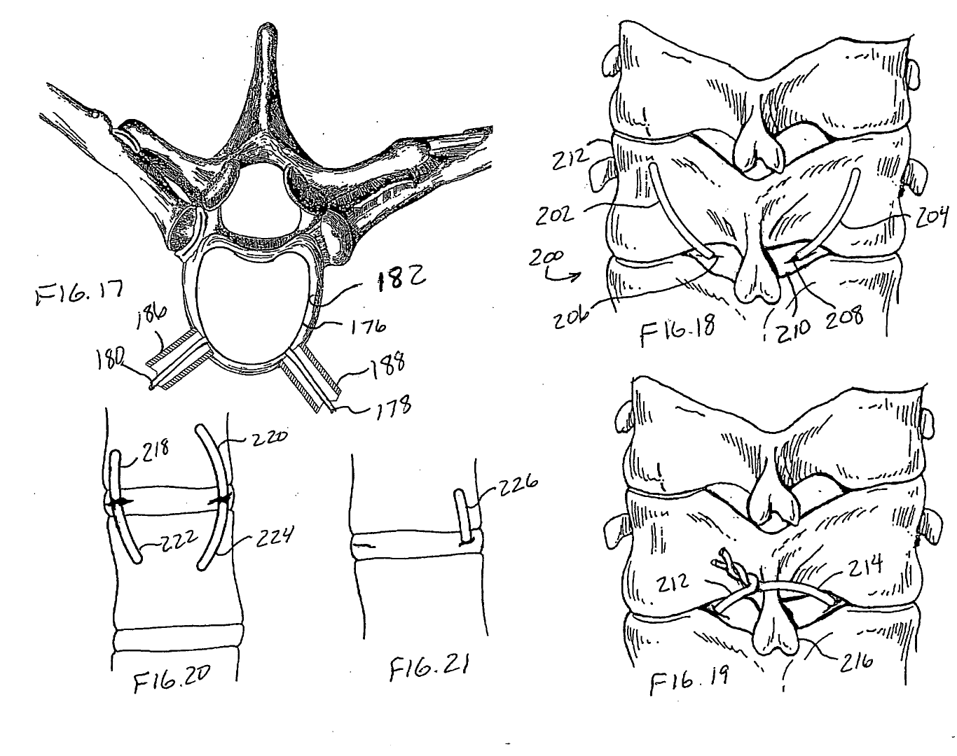

Alternatively, leads may be affixed to each other as shown in FIG. 19. In FIG. 19, the leads 212 and 214 are twisted together over the top of a bony protuberance 216. For leads made from a wire type material, simply twisting the leads together may provide the desired coupling. Alternatively, the leads may be coupled together using a knot, or coupled together using a screw, staple, rivet, glue or other device. Additionally, the filler device may be fixed using more or fewer than two leads. FIG. 20 depicts four leads 218, 220, 222 and 224 used to provide fixation while a single lead 226 is used in FIG. 21.

The use of a single lead to provide fixation may be facilitated by the use of incisions of different sizes. By way of example, FIG. 22 depicts an intervertebral disc 228. Two cannula 230 and 232 extend into a cavity 234 through the incisions 236 and 238, respectively, made in the annulus 240. The cannula 230 and 232 include inner bores 242 and 244, respectively. The bore 242 defines a larger diameter than the bore 244. Thus, the cannula 230 has a larger diameter than the cannula 232. Accordingly, the incision 236 is larger than the incision 238 to accommodate the larger diameter of the cannula 230.

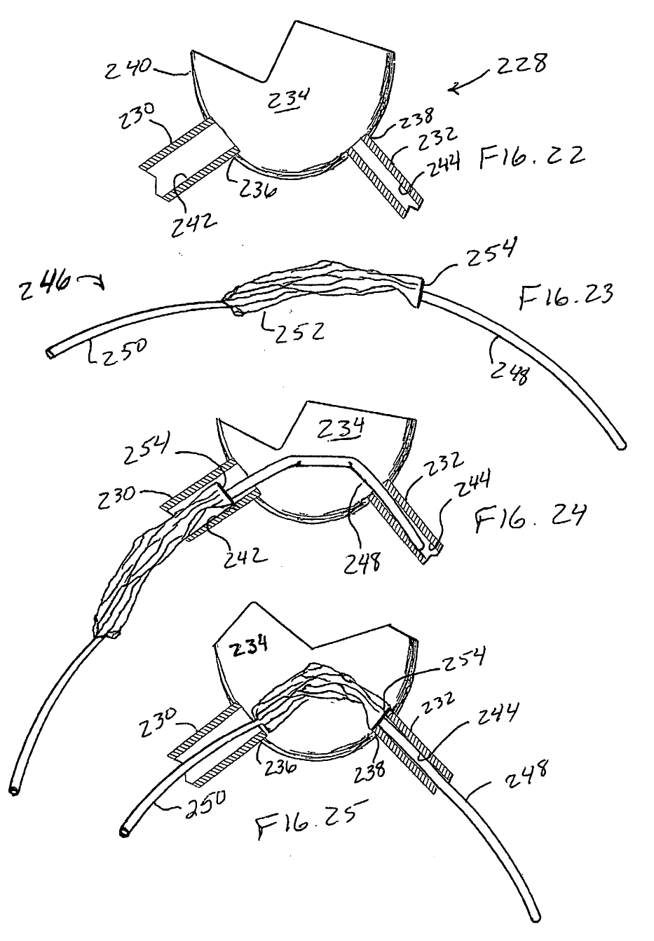

The filler device 246 shown in FIG. 23 is used with the configuration shown in FIG. 22. The filler device 246 includes two leads 248 and 250 and a deformable main body portion 252. The main body portion 252 includes a rigid disc portion 254. The lead 248 is sized to fit within both the inner bore 242 and the smaller diameter inner bore 244 as shown in FIG. 24.

The rigid disc portion 254 is also sized to fit within the inner bore 242, thus allowing the rigid disc portion 254 and the main body portion 252 to be positioned within the cavity 234 through the cannula 230. The rigid disc portion 254, however, has a diameter greater than the diameter of the inner bore 244. Thus, when the free end portion of the lead 248 is pulled through the inner bore 244 to the position shown in FIG. 25, the rigid disc portion 254 contacts the cannula 232 and maintains the main body portion 252 within the cavity 234. The contact between the rigid disc portion 254 and the cannula 232 provides an indication to the operator of the position of the filler device 246.

Additionally, in this embodiment the rigid disc portion 254 has a diameter greater than the diameter of the cannula 232. Therefore, unlike the filler device 174 which can be inserted with either lead 178 or 180 as the leading lead, the lead 248 is preferably the leading lead while the lead 250 is the trailing lead. This ensures that the main body portion 252 is not inadvertently positioned within the cannula 232.

The rigid disc portion 254 further has a diameter greater than the diameter of the incision 238. Thus, even when the cannula 232 has been removed, the rigid disc portion 254 is inhibited from moving through the incision 238. Accordingly, by fixing the lead 248 to a vertebra in the manner shown with respect to the lead 226 in FIG. 21, the filler device 246 is fixed within the cavity 234. The lead 250 may be removed or affixed to a bone or the lead 248 if additional fixation is desired. Alternatively, a filler device with a single lead may be used.

Many of the foregoing features may be combined. By way of example, a filler device may incorporate a body portion that is resiliently deformable and porous. Thus, after being deformed to allow for passage through a cannula, the body portion is allowed to return to a non-deformed shape within a cavity in an intervertebral disc. The body portion can then be used as a support structure for an in-situ curable filler material which can be injected into the body portion and extruded out of the pores to over-mold the body portion. If desired, the body portion may have regions of different porosity to allow for selective over-molding of the body portion. Support structures may be in the form of a mesh, a net, a bag or similar structure. The support structure may be deformable for ease of insertion into a cavity.

The preferred materials for use in the various embodiments may vary depending upon the particular configuration and method used. Thus, various components may be constructed from stainless steel, titanium, polymers, polyesters, or polyurethanes. Alternatively, various components may be made from rigid or compliant materials including stainless steel, titanium, memory metals, silicones, polyesters, polyurethanes, poly ether ether ketone (PEEK) or polypropylenes. Additionally, the materials may be used to deliver chemicals to the area in which the filler device is positioned. By way of example, but not of limitation, any of the various components may be imbedded or coated with a medication for relieving pain.

While the present invention has been illustrated by the description of exemplary processes and system components, and while the various processes and components have been described in considerable detail, the applicants do not intend to restrict or in any way limit the scope of the appended claims to such detail. Additional advantages and modifications will also readily appear to those ordinarily skilled in the art. The invention in its broadest aspects is therefore not limited to the specific details, implementations, or illustrative examples shown and described. Accordingly, departures may be made from such details without departing from the spirit or scope of the applicants' general inventive concept.

We claim:

1. A method of positioning an implant within a disc space of a patient comprising:
making a first incision in an annulus of an intervertebral disc;
making a second incision in the annulus of the intervertebral disc;
clearing a space within the intervertebral disc;
moving a first filler portion into the space through the first incision;
moving a second filler portion into the space through the first incision; and
moving the second filler portion out of the space through the second incision.

2. The method of claim 1, wherein:
moving the second filler portion into the space comprises injecting an in-situ curable material into the space; and
moving the second filler portion out of the space comprises extruding the in-situ curable material out of the space.

3. The method of claim 2, further comprising:
positioning a support structure within the space; and
over-molding the support structure with the in-situ curable material.

4. The method of claim 1, wherein moving the second filler portion out of the space comprises:
moving an end portion of a lead through the second incision.

5. The method of claim 4, wherein moving a first filler portion comprises:
pulling the end portion of the lead through the second incision to move the first filler portion into the space.

6. The method of claim 4, further comprising:
attaching the end portion of the lead to the patient.

7. The method of claim 4, further comprising:
coupling the end portion of the lead with a trailing lead portion connected to the first filler portion through the first incision.

8. The method of claim 1, wherein making a second incision in the annulus of the intervertebral disc comprises:
making a second incision having a size smaller than the size of the first incision.

9. The method of claim 8, further comprising:
moving a rigid filler portion into the space through the first incision; and
positioning the rigid filler portion within the space adjacent the second incision.

10. A method of positioning an implant within a disc space comprising:
making a first incision in an annulus of an intervertebral disc;
making a second incision in the annulus of the intervertebral disc;
clearing a space within the intervertebral disc;
moving a first filler portion into the space through the first incision; and
determining the position of the first filler portion using the second incision.

11. The method of claim 10, further comprising:
threading a first lead connected to the first filler portion through the second incision.

12. The method of claim 11, wherein determining the position of the first filler portion comprises:
identifying a mark on the first lead; and
correlating the mark with a distance between the mark and the first filler portion.

13. The method of claim 10, further comprising:
expanding the first filler portion within the space.

14. The method of claim 13, wherein expanding the first filler portion comprises:
injecting a fluid into the first filler portion through a lead extending through the first incision.

15. The method of claim 14, wherein injecting a fluid into the first filler portion comprises:
injecting a fluid into the first filler portion using a cannula placed through the first incision.

16. The method of claim 10, further comprising:
over-molding the first filler portion with an in-situ curable fluid.

17. The method of claim 10, wherein determining the position of the first filler portion comprises:
extruding a portion of the first filler portion out of the space through the second incision.

18. A device implanted within an intervertebral disc comprising:
a main body portion located within the intervertebral disc;
a first lead connected to the main body portion and extending out of the intervertebral disc through a first incision; and
a second lead connected to the main body portion and extending out of the intervertebral disc through a second incision.

19. The device of claim 18, wherein the first lead and the second lead are configured to be coupled to at least one vertebra located adjacent the intervertebral disc.

20. The device of claim 18, wherein the first lead and the second lead are configured to be coupled to each other at a location outside of the intervertebral disc.

21. The device of claim 18, wherein the main body portion comprises a first material, the first material deformable.

22. The device of claim 21, wherein the first material defines a porous structure, the main body portion further comprising:
a second material cured about the porous structure, the second material less deformable than the first material.

23. The device of claim 18, wherein the first lead comprises an inner bore in fluid communication with the main body portion.

24. The device of claim 18, wherein the main body portion comprises:
a rigid portion, the rigid portion sized to allow insertion of the rigid portion within a bore of a cannula inserted into the first incision and sized to not allow insertion of the rigid portion within a bore of a cannula inserted into the second incision.
